(19) United States
(12) Patent Application Publication
(10) Pub. No.: US 2016/0022953 A1
(43) Pub. Date: Jan. 28, 2016

(54) HEATING MEANS AND METHODS OF MANUFACTURE

(71) Applicant: SMITHS MEDICAL INTERNATIONAL LIMITED, Ashford, Kent (GB)

(72) Inventor: Fredrick M. Richards, Plymouth, MA (US)

(73) Assignee: Smiths Medical International Limited, Ashford, Kent (GB)

(21) Appl. No.: 14/771,864

(22) PCT Filed: Feb. 26, 2014
(86) PCT No.: PCT/GB2014/000065

§ 371 Date: Sep. 1, 2015

(30) Foreign Application Priority Data
Mar. 15, 2013 (GB) 1304691.7

(51) Int. Cl. A61M 16/10; A61M 16/08; H01C 17/06; H01C 17/28; H05B 3/34; H05B 3/14

(52) U.S. Cl. CPC A61M 16/1095; H05B 3/34; H05B 3/14; H01C 17/06; H01C 17/28; A61M 16/0875; A61M 2205/3653

(57) ABSTRACT

Heating elements for respiratory tubing (73) comprise an electrically-insulating substrate (22, 45, 50) with a printed looped track (21, 31, 41) of carbon extending along the substrate between two metal terminals (25, 26, 43, 44, 74, 75) at one end. The heating elements are preferably made by printing several looped tracks (51 to 59) side by side on an insulative substrate (50), applying a metal bus bar (60) along one edge (61) of the sheet over the free ends of the track loops. The bus bar is cut between the free ends of the tracks (51 and 59) to form isolated terminal pads connected with opposite ends of the tracks. The sheet is cut (63) between the tracks (51 to 59) to divide it into separate heating elements.

This invention relates to heaters of the kind including a resistive path of a conductive material extending between two terminals at opposite ends of the path by which a voltage can be applied across the path to produce a heating effect.

The invention is more particularly, but not exclusively, concerned with heaters and heating elements for use in respiratory circuits.

Respiratory humidifiers and nebulisers suffer from the problem that there is a tendency for some of the vapour produced by the apparatus to condense in the respiratory tubing circuit that conveys the vapour to the patient. The presence of condensate in either the inspiratory or expiratory breathing limbs is a problem because it presents a possible hazard if it should pass to the patient. It can also provide a site for the accumulation of bacteria, leading to a risk of infection. The need to remove and dispose of any collected condensate is also a problem. It is known that this “rain-out” can be reduced by heating the tubing so that the gas flowing along it is less likely to condense. This heating is usually achieved by means of a resistive metal wire extending within or outside the tube. Examples of previous heating arrangements are described in U.S. Pat. No. 6,078,730, U.S. Pat. No. 6,167,883 and U.S. Pat. No. 8,122,882. The cost of such wire heating elements is relatively high for a disposable component and it is difficult to provide wire heating elements with different heating characteristics at different locations so that the heating effect can be maximised where it is needed and power requirements minimised.

It is an object of the present invention to provide alternative heating means and methods of manufacture.

According to one aspect of the present invention there is provided a heater of the above-specified kind, characterised in that the resistive path is provided by a track of a non-metallic material on a substrate having an electrically-insulative surface.

The terminals may be located at the same end of the substrate. The conductive material of the preferably includes carbon, which may be printed on the substrate. The substrate may be of an electrically-insulative plastics material. The heater may be configured with different regions having a different heating effect. The different regions may be provided by regions of the track having different cross-sections or different resistivities. Alternatively, the heater may have a different number of tracks in the different regions.

According to a second aspect of the present invention there is provided a method of manufacturing heating elements comprising the steps of providing a planar substrate with an electrically-insulating surface, depositing on the surface a plurality of resistive tracks of an electrically-conductive, non-metallic material, each track being in the form of a loop where the closed ends of each loop are positioned towards one edge of the substrate and the two open ends of each loop are positioned towards the opposite edge of the substrate, providing an electrically-conductive terminal before or after deposition of the tracks, the terminal extending across the open ends of each track in electrical contact with the ends of each track, and subsequently cutting the substrate between adjacent tracks to form a plurality of separate heating elements.

The terminal may initially be in the form of a continuous bus bar, material of the bus bar being removed from between open ends of the same track is removed to isolate the open ends electrically from one another. The substrate is preferably of an electrically-insulative plastics material and the tracks include carbon.

According to a third aspect of the present invention there is provided a heating element made by a method according to the above second aspect of the present invention.

According to a fourth aspect of the present invention there is provided a sheet for use in manufacturing a plurality of heating elements, characterised in that the sheet includes a planar substrate having an electrically-insulative surface and two opposite edges, a plurality of resistive tracks of an electrically-conductive, non-metallic material extending over the surface, that each track is a loop with two open ends located adjacent one of the edges, that the closed end of each loop is located adjacent the opposite edge, and that an electrically-conductive terminal extends parallel to and adjacent the one edge in electrical contact with the open ends of each track.

The substrate is preferably of an electrically-insulative material and the tracks include carbon.

According to a fifth aspect of the present invention there is provided a heating element made from a sheet according to the above fourth aspect of the present invention.

According to a sixth aspect of the present invention there is provided a method of manufacturing a plurality of heating elements from a sheet according to the above fourth aspect of the invention in which the terminal is a continuous bus bar, characterised in that the method includes the steps of separating the different tracks from one another and interrupting the bus bar between the two open ends of each track either before or after separating the tracks from one another.

According to a seventh aspect of the present invention there is provided a heating element made by a method according to the above sixth aspect of the present invention.

According to an eighth aspect of the present invention there is provided a respiratory tube for conveying breathing gas to or from a patient including a heater according to the above one aspect of the present invention or a heating element according to the above third, fifth or seventh aspect of the present invention.

A heating element, a respiratory tube including a heating element and a method of manufacture of a heating element in accordance with the present invention will now be described, by way of example, with reference to the accompanying drawings, in which:

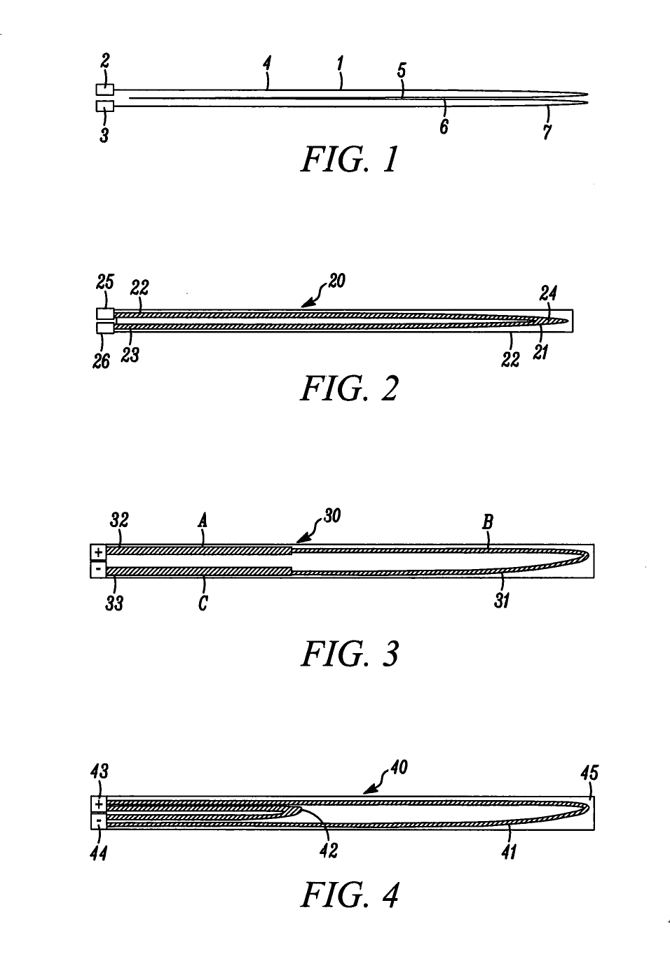

FIG. 1 is a plan view of a conventional, prior art heating element;

FIG. 2 is a plan view of a heating element of the present invention;

FIGS. 3 and 4 are plan views of alternative heating elements;

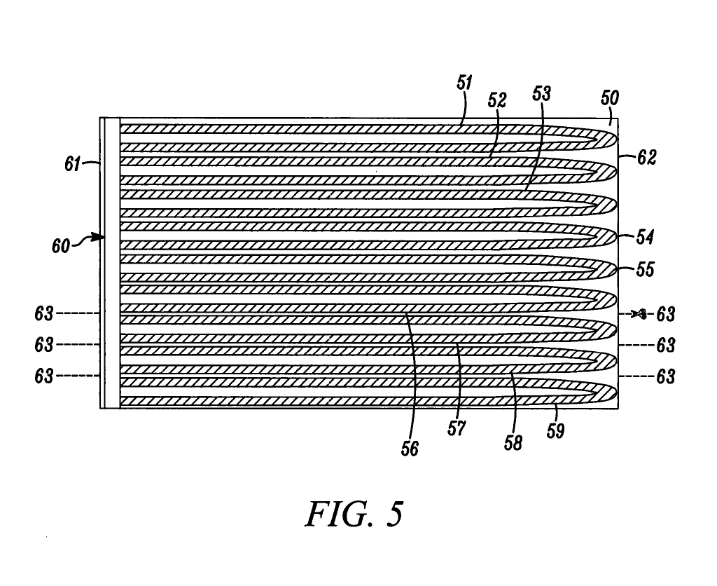

FIG. 5 is a plan view of a sheet from which nine heating elements can be formed; and

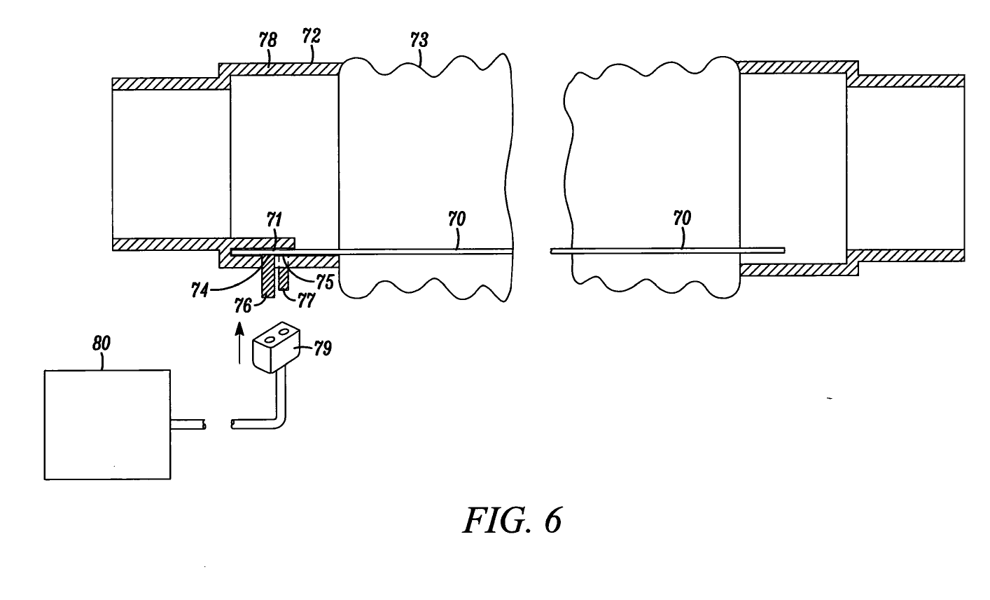

FIG. 6 is a cross-sectional side elevation view of a respiratory tube including a heating element.

With reference first to FIG. 1 there is shown a conventional heating element where a resistive path is provided by a length of an insulated, electrically-conductive resistance wire 1 made from a metal such as copper and joined at opposite ends to conductive terminal tabs 2 and 3. The wire 1 is formed into a loop and doubled back to form four sections 4 to 7 extending side by side so as to improve heat transfer to gas flowing along the respiratory tube within which it is inserted. The terminals 2 and 3 are located at the same end of the element so that they can be accessible from one end of the tube. In a typical respiratory tube with a length of about 150 cm, it can be seen that the heating element will need to have approximately 150×4=600 cm of wire. The high cost of copper makes the heating elements relatively expensive, especially for a component designed to be disposed of after a single use. It is desirable with respiratory breathing tubes to be able to provide the maximum heating effect in regions where this has most effect on reducing rain-out so as to produce the optimum effect for a given power input. It is, however, difficult to vary the heating effect along prior heating elements to produce the desired heating effect.

FIG. 2 shows a first example of heating means according to the present invention in the form of a heating element 20 provided by a non-metallic, electrically-conductive track deposited on an electrically-insulative surface of a planar substrate. More particularly, the element takes the form of a carbon track 21 printed on the surface of a rectangular polyester sheet 22 and formed into a loop having two free ends 22 and 23 at one end of the sheet and a closed end 24 at the opposite end of the sheet. The free ends 22 and 23 are in contact with respective metal terminal pads 25 and 26, such as of copper, overlaid on the ends 22 and 23 of the track. The track 21 need not be entirely of carbon but could be mixed with other materials to produce the desired resistivity. The cross-section of the carbon track 21 is selected to provide the desired resistance between the free ends, that is, by suitable selection of the width and/or thickness of the track. In general, the resistivity of a carbon track is higher than that of a copper wire so a carbon track will have a larger cross section than a wire of the same resistance. This means that the surface area of a printed carbon track will be larger than that of an equivalent copper wire so that the heat dissipation will be greater than that of a copper wire of the same length. In this way, the carbon track can be shorter than a copper wire with the same heat dissipation, thereby avoiding the need to double back the track in the manner of previous metal wires shown in FIG. 1. So, for the same length respiratory tube, a heating element according to the present invention requires only half the length of relatively inexpensive carbon compared with a doubled-back element of relatively expensive copper or other metal.

It will be appreciated that the resistive track needs to be supported on an electrically-insulative surface. This could either be provided by an insulative substrate, as described above, or the substrate could be conductive but have a layer of insulating material on which the track is deposited.

The heating means according to the present invention also enables heating circuits to be provided having a plurality of different temperature zones by appropriate variation in the characteristic of the deposited track along its length, as shown in FIG. 3. In this arrangement the carbon track 31 has the same shape as that shown in FIG. 2 but, instead of the track having the same cross section along, its length, the cross section and hence the resistivity of the track is different at different locations along the heating element 30. The track 31 shown in FIG. 3 has three different regions A, B and C along its length. The regions A and C extend along about one third of the length of the heating element from the free ends 32 and 33, whereas the region B extends between the two end regions providing the closed end of the loop. The track 31 in the regions A and C is relatively wide, that is, with a larger cross section, so has a relatively low resistivity; the region B is thinner, that is, with a smaller cross section, so it has a higher resistivity. The heating effect per unit length along the regions A and C, that is, towards the terminal end of the element 30, will be lower than the heating effect along the region B, that is, away from the terminal end. This heating element will, therefore be hotter away from the terminals than adjacent the terminals.

The heating effect along a heating element could be varied in other ways. For example, instead of using the same track material and varying its cross section (width and/or thickness), it would be possible to modify the heating effect by using different materials, that is, by depositing, non-metallic materials of different resistivity, at different regions along the track. These different materials could be printed or otherwise deposited one after the other. It will be appreciated that the heating effect could be varied by a combination of both variation in cross section and the use of materials of different resistivity.

Another configuration for modifying the heating effect along the length of a heating element is shown in FIG. 4. This shows a heating element 40 with two printed resistive tracks 41 and 42 connected with the two terminals 43 and 44 and connected in parallel with one another. One track 41 extends in a loop along the entire length of the substrate 45. The other track 42 is also formed in a loop and extends from the same terminals 43 and 44 within the longer track 41 but only extends along about one third the length of the heating element 40 at the terminal end of the element. This heating element 40 produces a greater heating effect in the region closer to the terminals 43 and 44 than in the region away from the terminals. The heating effect can be further modified by appropriate selection of the cross section or the materials for the two tracks, which may be the same or different. The heating element need not only have two tracks but could have three or more.

The heating elements of the present invention can be produced readily in the manner shown in FIG. 5. A rectangular sheet 50 of an insulative material such as polyester, or a conductive sheet with an insulative upper layer, is provided and nine resistive track loops 51 to 59 of a carbon-containing material are printed horizontally alongside one another on the upper surface of the sheet. The open end of each resistive track loop 51 to 59 is located adjacent the left-hand edge 61 of the sheet 50 and the closed end is located adjacent the opposite, right-hand edge 62 Any suitable carbon printing technique could be used such as ink jet printing, silk screen printing, photolithography or the like. Any number of two or more tracks could be printed. Next, terminal means in the form of a thin metal bus bar 60, such as of copper, is laid vertically down the left-hand edge 61 of the sheet 50 on top of the free ends of the tracks 51 to 59 so that these are all electrically bridged. The bus bar 60 could be formed in any conventional manner, such as by a metal foil bonded to the tracks 51 to 59 and to the substrate 50 by a conductive adhesive. Instead of applying the bus bar on top of the tracks it could be laid on the substrate before the tracks so that the tracks extend on top of the bus bar. The next step is to interrupt the bus bar 60 between the two free ends of each track 51 50 59 so that they are not electrically interconnected via the bus bar. This is done by removing a part of the bus bar 60 between the free ends of each track 51 to 59. Most conveniently this also involves removing the underlying part of the sheet 50, although this is not essential. The removal may be achieved by a mechanical method, such as milling, grinding or cutting, or by a chemical method, such as etching, or by some other technique such as laser ablation or cutting. The final step is simply to cut the sheet 50 horizontally between each track 51 to 59 eight times so as to divide it into nine separate and identical heating elements. FIG. 5 illustrates the lower three cut lines 63. The step of interrupting the bus bar between the free end of each track could be carried out after the sheet has been cut into separate elements instead of before this step, as described above.

The terminal means need not be laid on the sheet as a continuous bus bar but could, for example, be printed as a series of metal terminal pads separated from one another before or after printing the carbon tracks.

This manufacturing technique enables heating elements to be mass produced at low cost.

The heating element 70 is inserted along the bore of a respiratory tube 73 of the kind shown in FIG. 6, for inspiratory, expiratory or bidirectional use. The heating element 70 has its terminal end 71 located the machine end 72 of the tube 73 and the terminals 74 and 75 are electrically connected with respective contacts 76 and 77 extending through the wall of the machine end coupling 78. The contacts 76 and 77 connect with a mating connector 79 connected with a heater control unit 80 to deliver power to the heating element 70. The respiratory tube 73 may include temperature feedback from a sensor (not shown) connected in the breathing circuit to maintain a constant desired temperature of gas flowing along the tube.

1-18. (canceled)

19. A method of manufacturing heating elements comprising the steps of providing a planar substrate with an electrically-insulating surface, depositing on the surface a plurality of resistive tracks of an electrically-conductive, non-metallic material, each track being in the form of a loop where the closed ends of each loop are positioned towards one edge of the substrate and the two open ends of each loop are positioned towards the opposite edge of the substrate, providing an electrically-conductive terminal before or after deposition of the tracks, the terminal extending across the open ends of each track in electrical contact with the ends of each track, and subsequently cutting the substrate between adjacent tracks to form a plurality of separate heating elements.

20. A method according to claim 19, characterized in that the terminal is initially in the form of a continuous bus bar and that material of the bus bar is removed from between open ends of the same track to isolate the open ends electrically from one another.

21. A method according to claim 19, characterized in that the substrate is of an electrically-insulative plastics material.

22. A method according to claim 19, characterized in that the tracks include carbon.

23. A heating element made by a method comprising the steps of providing a planar substrate with an electrically-insulating surface, depositing on the surface a plurality of resistive tracks of an electrically-conductive, non-metallic material, each track being in the form of a loop where the closed ends of each loop are positioned towards one edge of the substrate and the two open ends of each loop are positioned towards the opposite edge of the substrate, providing an electrically-conductive terminal before or after deposition of the tracks, the terminal extending across the open ends of each track in electrical contact with the ends of each track, and subsequently cutting the substrate between adjacent tracks to form a plurality of separate heating elements.

24. A heating element according to claim 23, characterized in that heating element is configured with different regions having a different heating effect.

25. A heating element according to claim 24, characterized in that the different regions are provided by regions (A, B and C) of the track having different cross-sections or different resistivities.

26. A heating element according to claim 24, characterized in that the heating element has a different number of tracks in the different regions.

27. A sheet for use in manufacturing a plurality of heating elements, characterized in that the sheet includes a planar substrate having an electrically-insulative surface and two opposite edges, a plurality of resistive tracks of an electrically-conductive, non-metallic material extending over the surface, that each track is a loop with two open ends located adjacent one of the edges, that the closed end of each loop is located adjacent the opposite edge, and that an electrically-conductive terminal extends parallel to and adjacent the one edge in electrical contact with the open ends of each track.

28. A sheet according to claim 27, characterized in that the substrate is of an electrically-insulative plastics material and the tracks include carbon.

29. A heating element made from a sheet that comprises a planar substrate having an electrically-insulative surface and two opposite edges, a plurality of resistive tracks of an electrically-conductive, non-metallic material extending over the surface, that each track is a loop with two open ends located adjacent one of the edges, that the closed end of each loop is located adjacent the opposite edge, and that an electrically-conductive terminal extends parallel to and adjacent the one edge in electrical contact with the open ends of each track.

30. A method of manufacturing a plurality of heating elements from a sheet that includes a planar substrate having an electrically-insulative surface and two opposite edges, a plurality of resistive tracks of an electrically-conductive, non-metallic material extending over the surface, that each track is a loop with two open ends located adjacent one of the edges, that the closed end of each loop is located adjacent the opposite edge, and that an electrically-conductive terminal extends parallel to and adjacent the one edge in electrical contact with the open ends of each track, wherein the terminal is a continuous bus bar, characterized in that the method includes the steps of separating the different tracks from one another and interrupting the bus bar between the two open ends of each track either before or after separating the tracks from one another.

31. A heating element made by a method according to claim 30.

32. A respiratory tube for conveying breathing gas to or from a patient including a heating element made by a method comprising the steps of providing a planar substrate with an electrically-insulating surface, depositing on the surface a plurality of resistive tracks of an electrically-conductive, non-metallic material, each track being in the form of a loop where the closed ends of each loop are positioned towards one edge of the substrate and the two open ends of each loop are positioned towards the opposite edge of the substrate, providing an electrically-conductive terminal before or after deposition of the tracks, the terminal extending across the open ends of each track in electrical contact with the ends of each track, and subsequently cutting the substrate between adjacent tracks to form a plurality of separate heating elements.
